(19) United States
(12) Patent Application Publication
(10) Pub. No.: US 2014/0160427 A1
(43) Pub. Date: Jun. 12, 2014

(54) APPARATUS FOR MEASURING EYE MOVEMENT

(71) Applicant: Lucille M.J. Linde, Greeley, CO (US)

(72) Inventor: Lucille M.J. Linde, Greeley, CO (US)

(21) Appl. No.: 13/935,736

(22) Filed: Jul. 5, 2013

Related U.S. Application Data
(60) Provisional application No. 61/668,236, filed on Jul. 5, 2012.

(51) Int. Cl. A61B 3/113

(52) U.S. Cl. CPC A61B 3/113
USPC 351/203

(57) ABSTRACT

An apparatus for measuring eye movement (ocular pursuit) of a subject by moving a target along an arcuate member disposed ahead of the subject is described, wherein the target is moved in a channel formed in the arcuate member, and a clamp adapted for moving with the target for locating the position of the target along the arcuate member, thereby reducing unintended further movement thereof once the measurement has been completed.

### CROSS-REFERENCE TO RELATED APPLICATIONS

The present application claims the benefit of U.S. Provisional Patent Application No. 61/668,236 for “Apparatus For Measuring Eye Movement” filed on Jul. 5, 2012, the entire contents of which application is hereby specifically incorporated by reference herein for all that it discloses and teaches.

### FIELD OF THE INVENTION

The present invention relates generally to measurement of eye movement and, more particularly, to measurement of ocular tracking ability.

### BACKGROUND

In U.S. Pat. No. 4,818,097, entitled: “Ocular-Pursuit Monitoring,” which issued on Apr. 4, 1989 to Lucille M. J. Linde, and U.S. Pat. No. 5,048,947, entitled: “Process for Measuring Eye Movement,” which issued on Sep. 17, 1991 to Lucille M. J. Linde, a process for a quick, simple, objective, and accurate measurement of an individual's ocular tracking ability is described, including the use of an ocular-pursuit measuring instrument which comprises an arcuate member pivotally mounted at its midpoint to the upper end of a stand or leg member in a manner such that the arcuate member can be pivoted about an axis passing through the midpoint perpendicular to the axis of the stand or leg supporting the arcuate member. The arcuate member includes a graduated scale extending outward from the midpoint of the arcuate member in both directions. Ocular-pursuit measurements provide objective information indicative of the generally neurological health of the subject, and enables treatment of the subject by motor perceptual training.

A target is moved along the arcuate member from the midpoint as far as smooth eye movement by such person following the target is completed accurately; that is, when eye movement becomes irregular, and unable to focus on the target, the testing is repeated from a lower starting point, and the target is moved outwardly from the midpoint and then back again in successive increasing distances until the second point of nystagmus is observed. The number of units between the midpoint of the arcuate member and the last or maximum point where smooth eye movement was completed accurately are then recorded; that is, the farthest point of smooth eye movement is recorded. This process is used on each eye individually and on both eyes together to determine each subtest score. Subtest scores are totaled for a single score.

The person conducting the test moves the target along the arcuate member for the subject to follow with his or her eyes. The target is moved along the arcuate member by holding the target with one hand in a manner such that the target is in close proximity to the arcuate member. Although such procedures can be used effectively, the person holding and moving the target is required to avoid uneven movement of the target since such movements may interfere with the measurement, leading to a false test score.

Even in the situation where the target is supported at least in part on the arcuate member, thereby preventing upward and downward motion thereof, significant skill is required to prevent the target from being moved off of the arcuate member since the person conducting the test is closely following the eyes of the subject.

### SUMMARY OF THE INVENTION

Embodiments of the present invention overcome the disadvantages and limitations of the prior art by providing an apparatus for measuring eye movement, wherein the target is prevented from being dislodged from the measuring apparatus.

Another object of embodiments of the invention is to provide an apparatus for measuring eye movement, wherein the target position is located once its movement is ceased.

Additional objects, advantages and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

To achieve the foregoing and other objects, and in accordance with the purposes of the present invention, as embodied and broadly described herein, the apparatus for measuring eye movement of a subject (ocular pursuit), hereof, includes: an open arcuate member having a chosen length and curvature, a channel formed through a portion of the length, and an upper surface into which one side of the channel opens, the arcuate member being disposed before the subject such that the arcuate member opens towards the subject; a stand for supporting said arcuate member; a target including a support member having a flat surface adapted for being moved along the upper surface of the arcuate member; an upper member disposed above the support member, attached thereto, and observable by the subject; a central portion attached to the support member adapted to be slidably moved in the channel of the arcuate member, and a stabilizing member, attached to the second portion and disposed below the arcuate member; and a clamp adapted for moving with the target for fixing the position of the target along the arcuate member and reducing unintended further movement thereof once the measurement has been completed.

Benefits and advantages of embodiments of the present invention include, but are not limited to, providing an apparatus for measuring eye movement where there is no need for the examiner to communicate with the subject being examined other than to request that the subject follow the target with his or her eyes, nor is there a requirement that the subject operate the apparatus. Since motion of the target is constrained to the channel in the arcuate member, the examiner is free to observe the eye movement of the subject without concern that the target will fall from the apparatus, thereby permitting an accurate and rapid determination of the location on the arcuate member where smooth eye movement is interrupted by erratic movement or inability to focus on a target.

### BRIEF DESCRIPTION OF THE DRAWINGS

The accompanying drawings, which are incorporated in and form a part of the specification, illustrate the embodiments of the present invention and, together with the description, serve to explain the principles of the invention. In the drawings:

FIG. 1 is a perspective view of an embodiment of the apparatus of the present invention, illustrating the channel cut through an arcuate member in which the target is moved, the target and the clamp for locating the point along the arcuate member where smooth eye movement is interrupted.

FIG. 2 is a side elevation view of the apparatus shown in FIG. 1 hereof, illustrating the target disposed in the channel of the arcuate member.

FIG. 3a is side view of the target shown in FIG. 1, while FIG. 3b is a perspective view thereof.

### DETAILED DESCRIPTION OF THE INVENTION

In the use of the apparatus described in the '097 and '947 patents, supra, the examiner requests that the subject follow a target with his or her eyes while the examiner moves the target along the arcuate member away from the midpoint. This process may become rapid and efficient with practice, but it was noted by the present inventor that occasionally when moving the target out to the periphery area, it would slip off the arcuate member. This necessitated either slowing the movement of the target, or observing the location of the target relative to the arcuate member, thereby taking the attention of the examiner away from the eyes of the subject.

Briefly, embodiments of the present invention include apparatus for measuring eye movement of a subject by moving a target along an arcuate member disposed ahead of the subject, wherein the target is moved in a channel formed in the arcuate member, and a clamp adapted for moving with the target for fixing (locating) the position of the target along the arcuate member and reducing unintended further movement thereof once the measurement has been completed. These improvements prevent the target from falling off of the arcuate member, requiring that the measurement be repeated, and permit the target location along the arcuate member to be measured without the necessity of the examiner having to closely watch the target movement, thereby permitting the examiner to observe the eyes of the subject being examined. The improvements both increase the accuracy of the measurements and reduce the time required.

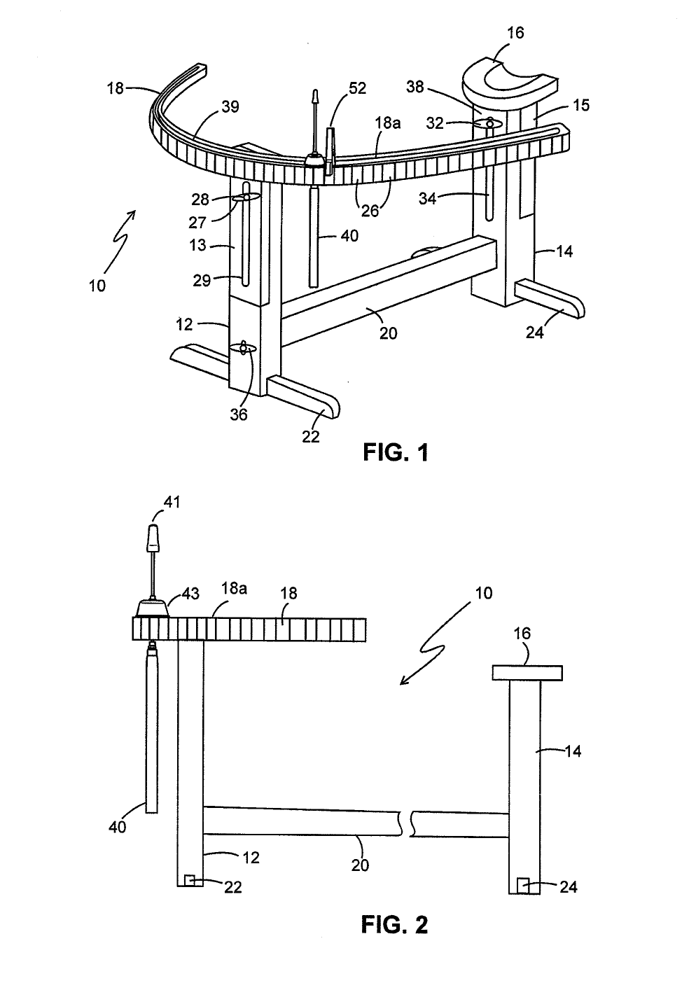

Reference will now be made in detail to the present embodiments of the invention, examples of which are illustrated in the accompanying drawings. In the FIGURES, similar structure will be identified using identical reference characters. It will be understood that the FIGURES are presented for the purpose of describing particular embodiments of the invention and are not intended to limit the invention thereto. Turning now to FIG. 1, a perspective view of an embodiment of apparatus, 10, of the present invention, which can be used in measurements of eye movement of a subject, is shown. First upstanding leg member, 12, and space-apart second upstanding leg member, 14, are connected to bar, 20, which securely holds leg members 12 and 14 in their relative positions. Leg member 12 includes foot member, 22, and leg member 14 includes foot member, 24, which are attached to the leg members for providing stability. Leg member 12 can be detached from bar 20 by removing nut, 36, from a threaded bolt extending through leg 12 and into bar 20, with a similar nut and bolt attachment at the opposite end of bar 20 to leg member 14.

Vertically adjustable member, 15, attached to chin support, 16, and movably attached leg member 14 at the upper end thereof permits apparatus 10 to accommodate individuals having different heights. The test subject places his or her chin on chin support 16 such that the subject's head is stabilized during measurements. Member 15 may be adjusted to an appropriate height using bolt, 32, attached to member 15 at one end, the other end thereof protruding through vertical slot, 34, in leg member 14, and wing nut, 38, which may be loosened to effectuate raising or lowering chin support 16.

Open arcuate member, 18, having flat upper surface, 18a, is pivotably attached to at its midpoint to the upper end of leg member 12. A bolt (not shown in FIG. 1) is secured at one end to arcuate member 18 and extends through leg member 12. Arcuate member 18 may be pivoted about its midpoint in one direction or the other for various testing procedure, and may be mounted either outside of leg member 12 as shown in FIG. 1, or inside thereof. In other embodiments, arcuate member 18 may be vertically adjusted. As an example, arcuate member 18 may be attached to member, 13, having vertical slot, 29, therein. Wing nut, 27, adapted to screw onto bolt, 28, extending through member 13 and leg member 12 may be loosened for raising a lowering arcuate member 18. Arcuate member 18 further includes a graduated scale along its periphery which is readable by the person conducting the examination, and has chosen graduation, 26. A tape measure secured to the front face of arcuate member 18, and having one-half inch graduations has been effectively used for the measurements of the present invention. Although the curvature of arcuate member 18 is a matter of choice, a radius in the range of about 20 in. has been found to be effective for a semi-circular arcuate member.

Pass-through channel, 39, in arcuate member 18 is adapted to receive target, 40, which may be smoothly moved therein during the measurement, and will be described in detail hereinbelow. Clamp, 52, for locating the point along the arcuate member where smooth eye movement is interrupted, is movably disposed in the pass through channel, and will also be described in more detail hereinbelow.

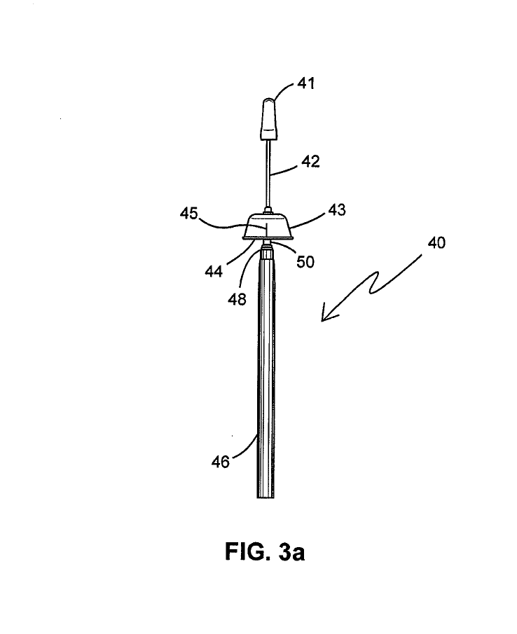

FIG. 2 is a side elevation view of the apparatus shown in FIG. 1 hereof, illustrating target 40 disposed in channel 39 of arcuate member 18.

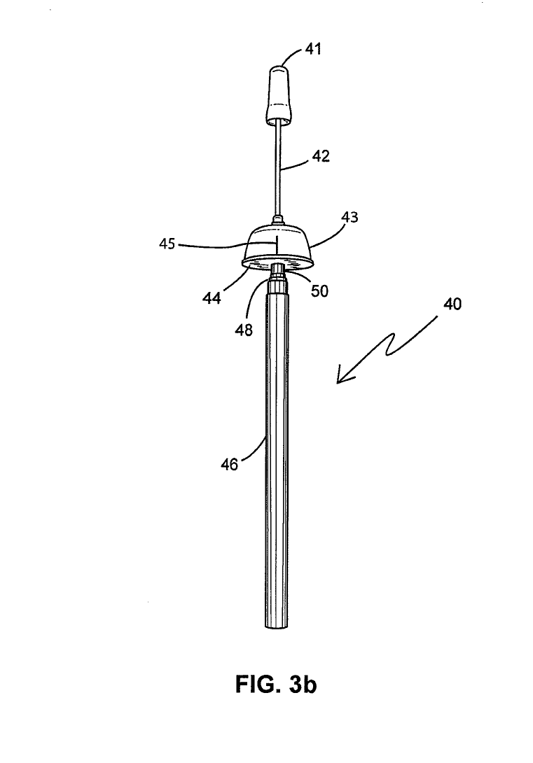

Turning now to the description of target 40, FIG. 3a is side view of target 40, while FIG. 3b is a perspective view thereof. Colored tip, 41, is secured to the upper end of stem, 42, the lower end of stem 42 being affixed to support member, 43, having lower flat surface, 44, adapted for being smoothly moved along upper surface 18a of arcuate member 18, as illustrated in FIGS. 1 and 2 hereof. Stabilizing member, 46, having female threaded portion, 48, adapted to be screwed together with threaded rod, 50, attached to support member 43 at bottom surface 44 thereof, forming a central portion, is adapted to smoothly fit through pass-through channel 39 in arcuate member 18, such that support member 43 and stabilizing member 46 maintain a vertical orientation of tip 41, and permit target 40 to be smoothly moved in channel 39.

Lower flat surface 44 of support member 43 is made sufficiently large that its contact with surface 18a of arcuate member 18 prevents it from tilting in the direction of movement, and keeps stem 42 approximately perpendicular to surface 18a. Support member 43 may also have vertical line, 45, inscribed therein perpendicular to surface 44 to more accurately indicate the position on scale 26 for the score.

For measuring eye movement of a subject it is necessary for the subject to place his or her chin on chin support 16 facing arcuate member 18. The examiner then requests that the subject follow colored tip 41 of target 40 with his or her eyes as the target is moved along arcuate member 18 away from the midpoint. When measuring the subject's ocular pursuit, a notation is made of the distance to the nearest one-half inch that tip 41 has been moved from the midpoint of arcuate member 18 while being followed by the eye with smooth eye movement. The measurement is then repeated moving target 40 away from the midpoint of arcuate member 18 in the opposite direction. The measurements may be performed with each of the subject's eyes individually, and with both eyes together.

Stabilizing member 46 and support member 43, of target 40 constrain target 40 to move along surface 18a of arcuate member 18. One leg of clamp 52, an example of which is a clothespin, is placed in channel 39 on face 18a of arcuate member 18 on the side of target 40 away from the midpoint of arcuate member 18. The other leg of clamp 52 slides along scale 26 of arcuate member 18. As target 40 is moved away from the midpoint in channel 39 during a measurement, clamp member 52 is driven in front of it. When the examiner ceases moving target 40, clamp member 52 provides a location on scale 26 of arcuate member 18 which may also be recorded. When the measurement is repeated, clamp member 52 is returned along with target 40 to the starting point. Clamp 52 may be placed on the opposite side of clamp 40 when motion thereof is intended in the other direction.

The improvements of the present invention keep support member 43 of target 40 in contact with face 18a of arcuate member 18, when target 40 is placed in channel 39, and prevent target 40 from being dislodged from arcuate member 18 and/or otherwise being moved away therefrom during a measurement. Measurements may then be made more efficiently and accurately since, not only can the examiner to maintain focus on the eyes of the subject being examined, but smooth motion of the target is possible, thereby reducing the possibility of erroneous scores.

Additional details concerning the operation of apparatus 10 and data resulting from measurements therewith may be found in U.S. Pat. No. 4,818,097, entitled: “Ocular-Pursuit Monitoring,” which issued on Apr. 4, 1989 to Lucille M. J. Linde, and U.S. Pat. No. 5,048,947, entitled: “Process for Measuring Eye Movement,” which issued on Sep. 17, 1991 to Lucille M. J. Linde, which patents are hereby incorporated herein in their entirety for all that they disclose and teach.

The foregoing description of the invention has been presented for purposes of illustration and description and is not intended to be exhaustive or to limit the invention to the precise form disclosed, and obviously many modifications and variations are possible in light of the above teaching. The embodiments were chosen and described in order to best explain the principles of the invention and its practical application to thereby enable others skilled in the art to best utilize the invention in various embodiments and with various modifications as are suited to the particular use contemplated. It is intended that the scope of the invention be defined by the claims appended hereto.

What is claimed is:

1. An apparatus for measuring eye movement of a subject, comprising:
an open arcuate member having a chosen length and curvature, a channel formed through a portion of the length, and an upper surface into which one side of the channel opens, said arcuate member being disposed before said subject such that said arcuate member opens towards said subject;
a stand for supporting said arcuate member;
a target comprising a support member having a flat surface adapted for being moved along the upper surface of said arcuate member; an upper member disposed above said support member, attached thereto, and observable by said subject; a central portion attached to said support member adapted to be slidably moved in the channel of said arcuate member; and a stabilizing member, attached to said central portion and disposed below said arcuate member; and
a clamp adapted for moving with said target for fixing the position of said target along said arcuate member and reducing unintended further movement thereof once the measurement has been completed.

2. The apparatus of claim 1, wherein the height of said stand is adjustable.

3. The apparatus of claim 1, wherein said stand further comprises a chin support for said subject.

4. The apparatus of claim 1, wherein said stabilizing member is effective for vertically orienting said upper member.

5. The apparatus of claim 4, wherein said upper member comprises a rod.

6. The apparatus of claim 5 wherein said rod comprises a colored tip.

7. The apparatus of claim 1, wherein said arcuate member is pivotable on said stand.

8. The apparatus of claim 1, wherein a portion of said clamp is adapted to move within the channel of said arcuate member.

9. The apparatus of claim 8, wherein said clamp comprises a clothespin.

10. The apparatus of claim 1, wherein said arcuate member comprises scale graduations.
